(19) United States
(12) Patent Application Publication
(10) Pub. No.: US 2026/0334830 A1
(43) Pub. Date: Oct. 29, 2026

(54) INFORMATION PROCESSING APPARATUS, INFORMATION PROCESSING SYSTEM, AND INFORMATION PROCESSING PROGRAM

(71) Applicant: SONY GROUP CORPORATION, TOKYO (JP)

(72) Inventor: MAO KATSUHARA, TOKYO (JP)

(21) Appl. No.: 19/102,493

(22) PCT Filed: Aug. 7, 2023
(86) PCT No.: PCT/JP2023/028705

§ 371 Date: Feb. 10, 2025

(30) Foreign Application Priority Data
Aug. 18, 2022 (JP) 2022-130794

(51) Int. Cl. A61B 5/16; A61B 5/256; A61B 5/374; A61B 5/378; A61B 5/38; A61B 5/384

(52) U.S. Cl. CPC A61B 5/165; A61B 5/256; A61B 5/374; A61B 5/378; A61B 5/38; A61B 5/384

(57) ABSTRACT

An information processing apparatus according to an embodiment of the present disclosure includes: an acquisition unit configured to acquire a first signal related to a brain wave of a user; an analysis unit configured to detect a specified frequency of the brain wave of the user on the basis of the first signal; and a control unit configured to control content on the basis of the specified frequency.

### TECHNICAL FIELD

The present disclosure relates to an information processing apparatus, an information processing system, and an information processing program.

### BACKGROUND ART

An apparatus has been proposed that determines a state of a subject using a brain wave signal of the subject and applies a stimulus by applying a current to a surface of a head of the subject in accordance with a result of the determination (PTL 1).

### CITATION LIST

### Patent Literature

PTL 1: Japanese Unexamined Patent Application Publication No. 2018-68511

### SUMMARY OF THE INVENTION

It is desired that a stimulus application apparatus be able to apply an appropriate stimulus to a user.

It is desirable to provide an information processing apparatus that makes it possible to apply a suitable stimulus.

An information processing apparatus according to an embodiment of the present disclosure includes: an acquisition unit configured to acquire a first signal related to a brain wave of a user; an analysis unit configured to detect a specified frequency of the brain wave of the user on the basis of the first signal; and a control unit configured to control content on the basis of the specified frequency.

An information processing system according to an embodiment of the present disclosure includes an electronic apparatus and an information processing apparatus. The electronic apparatus includes a sensor configured to generate a first signal related to a brain wave of a user. The information processing apparatus includes: an analysis unit configured to detect a specified frequency of the brain wave of the user on the basis of the first signal; and a control unit configured to control content on the basis of the specified frequency.

An information processing program according to an embodiment of the present disclosure causes a computer to: acquire a first signal related to a brain wave of a user; detect a specified frequency of the brain wave of the user on the basis of the first signal; and control content on the basis of the specified frequency.

### BRIEF DESCRIPTION OF THE DRAWINGS

FIG. 1 is a diagram illustrating an example of an overall configuration of an information processing system according to an embodiment of the present disclosure.

FIG. 2 is an explanatory diagram of a configuration example of the information processing system according to the embodiment of the present disclosure.

FIG. 3 is a flowchart illustrating an example of an operation of the information processing system according to the embodiment of the present disclosure.

FIG. 4 is a flowchart illustrating another example of the operation of the information processing system according to the embodiment of the present disclosure.

FIG. 5 is an explanatory diagram of a configuration example of an information processing system according to Modification Example 1 of the present disclosure.

FIG. 6 is an explanatory diagram of a configuration example of an information processing system according to Modification Example 2 of the present disclosure.

FIG. 7 is an explanatory diagram of a configuration example of an information processing system according to Modification Example 3 of the present disclosure.

FIG. 8 is an explanatory diagram of a configuration example of an information processing system according to Modification Example 4 of the present disclosure.

### MODES FOR CARRYING OUT THE INVENTION

Hereinafter, detailed description is given of an embodiment of the present disclosure with reference to the drawings. It is to be noted that the description is given in the following order.
1. Embodiment
2. Modification Examples
   2-1. Modification Example 1
   2-2. Modification Example 2
   2-3. Modification Example 3
   2-4. Modification Example 4
   2-5. Modification Example 5
   2-6. Modification Example 6

### 1. EMBODIMENT

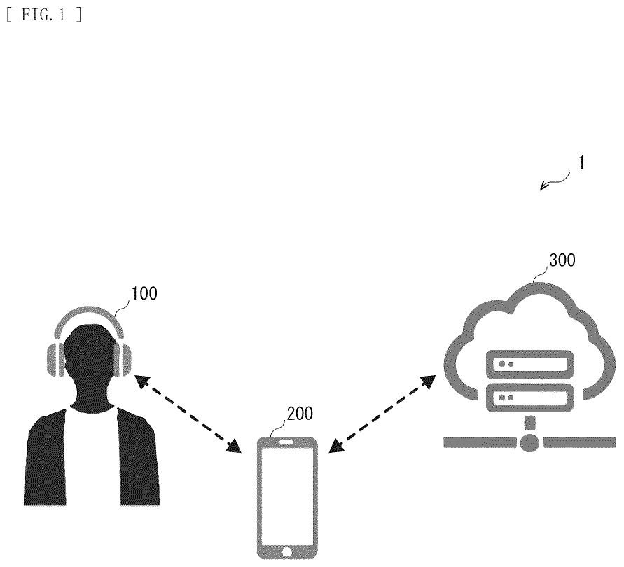

FIG. 1 is a diagram illustrating an example of an overall configuration of an information processing system according to an embodiment of the present disclosure. An information processing system 1 includes an electronic apparatus 100, an information processing apparatus 200, and a server 300 (e.g., a cloud server). The electronic apparatus 100 is a wearable apparatus and is an electronic apparatus worn by a user in use. The electronic apparatus 100 is a wearable device such as a headphone, an earphone, or eyewear. The electronic apparatus 100 may be, for example, a headphone or a VR goggle.

The information processing apparatus 200 is a terminal device (a terminal) to be used by the user. The information processing apparatus 200 is an electronic apparatus such as a smartphone, a tablet terminal, or a computer. The information processing apparatus 200 is configured to be coupled to a network and communicate with an external apparatus (such as the server 300). The electronic apparatus 100 and the information processing apparatus 200 may perform transmission and reception of information through wireless communication or wired communication. The electronic apparatus 100 may also be referred to as an edge device (an edge apparatus). Although described later, the electronic apparatus 100 includes a sensor configured to acquire a brain wave.

The information processing system 1 acquires a brain wave of the user with the electronic apparatus 100 and uses the acquired brain wave to control content (such as picture content or audio content) to be presented to the user. The information processing system 1 may promote a change in emotion of the user by applying a stimulus (such as a visual stimulus or an auditory stimulus) to the user using the content. The application of the stimulus to the user using the content enables a change in the emotion and control of the emotion.

It is to be noted that the information processing apparatus 200 and the electronic apparatus 100 may also be collectively referred to as an information processing apparatus. Further, the information processing apparatus 200 and the external apparatus that are coupled to each other via the network may also be collectively referred to as an information processing apparatus.

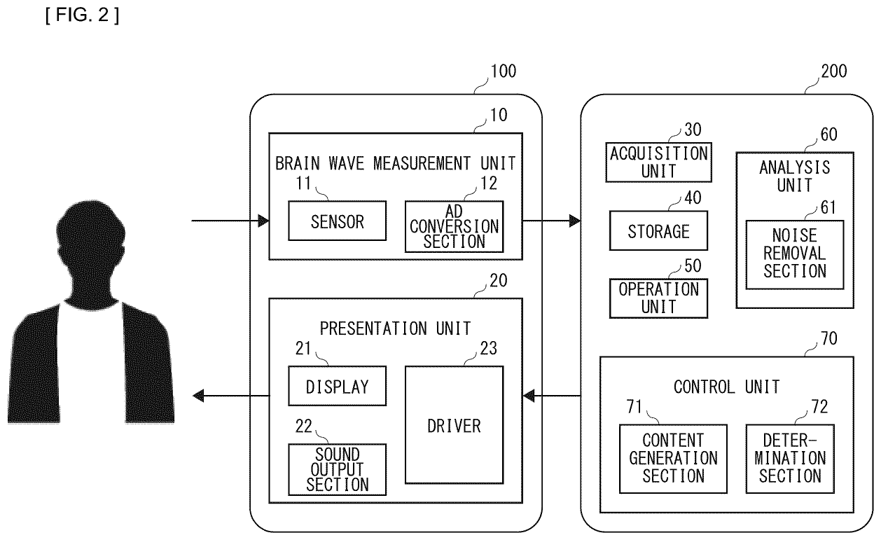

FIG. 2 is an explanatory diagram of a configuration example of the information processing system according to the embodiment. In the example illustrated in FIG. 2, the electronic apparatus 100 includes a brain wave measurement unit 10 and a presentation unit 20. It is to be noted that the electronic apparatus 100 includes an unillustrated communication unit including a communication module and the like and is configured to communicate with an external apparatus (such as the information processing apparatus 200).

As illustrated in FIG. 2, the brain wave measurement unit 10 includes a sensor 11 and an AD conversion section 12, and is configured to measure the brain wave. The brain wave measurement unit 10 is configured to generate a signal related to the brain wave (hereinafter, referred to as a brain wave signal). The brain wave signal is a brain wave or a signal including the brain wave, and may be, for example, a brain wave converted into a digital signal. The brain wave signal is an electrical signal corresponding to an activity state of a brain of the user as a living body.

The brain wave measurement unit 10 may measure the brain wave of the user and generate a brain wave signal. In the information processing system 1, the electronic apparatus 100 including the brain wave measurement unit 10 is worn by the user, allowing for detection of the brain wave (the brain wave signal). It is to be noted that the sensor 11 and the AD conversion section 12 may be integrally formed.

The sensor 11 is mounted on the electronic apparatus 100 such as a headphone and is configured to measure the brain wave. The sensor 11 is a brain wave sensor and is configured to generate a brain wave signal. The brain wave signal is, for example, a signal indicating a potential (a voltage) accompanied by an activity of the brain. The sensor 11 acquires the brain wave signal of the living body and outputs the acquired brain wave signal to the AD conversion section 12. The information processing system 1 acquires the brain wave signal, enabling estimation of a state of the living body.

As an example, the sensor 11 includes a plurality of electrodes (bioelectrodes) and an AFE (Analog Front End) section that are adapted to detect a potential, and is configured to measure a biopotential. The sensor 11 acquires, with the plurality of bioelectrodes and the AFE section, the brain wave signal serving as a signal that is based on a potential of a head of the user.

For example, the plurality of bioelectrodes of the sensor 11 includes an electrically conductive material and is brought into contact with the head of the user at respective positions different from each other. The bioelectrodes of the sensor 11 are brought into contact with a measurement site and provided with a potential of the contact site. The bioelectrodes are coupled to the AFE section and supply the AFE section with a signal corresponding to the potential of the site of the living body in contact therewith. The AFE section includes an amplifier circuit configured to amplify a signal. The AFE section may amplify the signal acquired by the bioelectrodes and output the amplified signal as the brain wave signal to the AD conversion section 12.

The AD conversion section 12 is configured to convert an inputted analog signal into a digital signal. The AD conversion section 12 is an ADC (Analog to Digital Converter). The AD conversion section 12 performs AD conversion processing on the brain wave signal as an analog signal inputted from the sensor 11. For example, the AD conversion section 12 converts the inputted brain wave signal into a digital signal of a predetermined number of bits. The electronic apparatus 100 may transmit, to the information processing apparatus 200 illustrated in FIG. 2, the brain wave signal converted into the digital signal by the AD conversion section 12.

The presentation unit 20 of the electronic apparatus 100 is configured to present the user with the content. The presentation unit 20 may also be referred to as a provision unit configured to provide the content. The presentation unit 20 may present (provide) the user with picture content, musical content, or the like. As illustrated in FIG. 2, the presentation unit 20 includes: a display 21 configured to display an image; a sound output section 22 configured to output a sound; and a driver 23.

The driver 23 is a drive section, and is configured to drive the display 21 and the sound output section 22. The driver 23 controls driving of the display 21 on the basis of image data. The driver 23 also controls driving of the sound output section 22 on the basis of audio data. The driver 23 may also be referred to as a control section configured to control the display 21 and the sound output section 22.

The display 21 is configured to display an image on the basis of the image data. The display 21 is a liquid crystal display, an organic EL display, or the like. It is to be noted that the display 21 may include a touch panel. The sound output section 22 is configured to output a sound that is based on the audio data. The sound output section 22 is a speaker or the like. The sound output section 22 may also be referred to as a conversion section that converts the audio data serving as an electrical signal into a sound (a sound wave). The sound output section 22 may output music (BGM), sound effects, or the like on the basis of the audio data.

The driver 23 may perform processing of causing the display 21 to display an image corresponding to the image data of the picture content. For example, the driver 23 controls the display 21 in accordance with the image data transmitted from the information processing apparatus 200, and causes the display 21 to display an image corresponding to the image data. The driver 23 may also perform processing of causing the sound output section 22 to output a sound corresponding to the audio data (an audio signal) of the audio content. For example, the driver 23 controls the sound output section 22 in accordance with the audio data transmitted from the information processing apparatus 200, and causes the sound output section 22 to output a sound corresponding to the audio data.

As described above, the image data to be displayed and the audio data to be reproduced are inputted into the presentation unit 20 from the information processing apparatus 200 or the like. The presentation unit 20 displays an image corresponding to the image data. The presentation unit 20 also generates a sound corresponding to the audio data. This allows the user to view or listen to the picture content or the audio content presented by the presentation unit 20. It is to be noted that some or all of the driver 23, the display 21, and the sound output section 22 may be integrally formed.

The information processing apparatus 200 includes a processor and a memory (such as a ROM or a RAM), and is configured to perform various kinds of information processing. The information processing apparatus 200 reads and executes a program incorporated therein, and performs information processing (signal processing). The information processing apparatus 200 may also serve as a controller, and may control the electronic apparatus 100 by performing communication of transmission and reception of information with the electronic apparatus 100 on the basis of the program.

In the example illustrated in FIG. 2, the information processing apparatus 200 includes an acquisition unit 30, a storage 40, an operation unit 50, an analysis unit 60, and a control unit 70. It is to be noted that the information processing apparatus 200 includes an unillustrated communication unit including a communication module and the like, and is configured to communicate with an external apparatus (such as the electronic apparatus 100 or the server 300).

The acquisition unit 30 is configured to acquire the brain wave signal related to the brain wave. In the example illustrated in FIG. 2, the acquisition unit 30 acquires the brain wave signal from the electronic apparatus 100 through communication with the electronic apparatus 100. The brain wave signal is generated regularly or irregularly by the electronic apparatus 100 including the sensor 11 and is outputted to the acquisition unit 30. The acquisition unit 30 may also be referred to as an input unit into which the brain wave signal is to be inputted.

The storage 40 includes a nonvolatile memory and holds (records) programs and data. The storage 40 stores various kinds of information such as programs and data to be used to control respective components of the information processing apparatus 200 or programs and data to be used for various applications. The storage 40 is a recording medium such as a semiconductor memory or a hard disk.

The operation unit 50 includes, for example, a touch panel or a keyboard, and is configured to receive an operation performed on the information processing apparatus 200. In accordance with an operation performed by the user, the operation unit 50 is configured to output a signal (an operation signal) that is based on the operation. The operation unit 50 may output the operation signal for various functions of the information processing apparatus 200. Further, it is possible for the user to perform various kinds of control of the information processing apparatus 200 and the electronic apparatus 100 using, as a user interface, an application on the information processing apparatus 200, for example.

The analysis unit 60 is configured to analyze the brain wave (the brain wave signal). The analysis unit 60 performs various kinds of signal processing on the brain wave signal acquired by the acquisition unit 30. Examples of the various kinds of signal processing include noise reduction processing, frequency analysis processing, and phase analysis processing. The analysis unit 60 may also be referred to as a signal processing unit configured to execute signal processing.

The analysis unit 60 includes a noise removal section 61 and is configured to execute processing of removing a signal component in an unnecessary band of the brain wave signal, noise removal processing, and the like. The analysis unit 60 may perform, for example, filter processing such as band-pass filter processing or notch filter processing and noise removal processing such as processing of removing noise caused by body motion or blinking. The analysis unit 60 performs the filter processing, the noise removal processing, and the like on the brain wave signal and removes (reduces) the unnecessary signal component mixed into the brain wave signal.

As described above, the analysis unit 60 is configured to execute frequency analysis processing on the brain wave signal. The analysis unit 60 performs frequency analysis processing on the brain wave signal having been subjected to the noise removal by the noise removal section 61. The analysis unit 60 calculates signal strength for each frequency by performing, for example, an FFT (Fast Fourier Transform (fast Fourier transform)) on the brain wave signal, and calculates a power spectrum (distribution).

The brain wave is classified according to a frequency band into, for example, an alpha wave, a beta wave, or a theta wave. The alpha wave ranges from 8 Hz to 13 Hz, the beta wave ranges from 13 Hz to 30 Hz, and the theta wave ranges from 4 Hz to 8 Hz. The brain wave signal includes a component of the alpha wave, a component of the beta wave, a component of the theta wave, and the like. The analysis unit 60 performs signal processing on the brain wave signal and thereby acquires a power spectrum representing a signal level at each frequency of the brain wave of the user. In the power spectrum, the signal strength (power) in each frequency range is a value corresponding to the activity state of the brain.

The analysis unit 60 is configured to detect a specified frequency of the brain wave on the basis of the brain wave signal. The analysis unit 60 performs processing of detecting a specified frequency fp of the brain wave using, for example, the calculated power spectrum. This can also mean that the analysis unit 60 analyzes the power spectrum and thereby extracts the specified frequency fp. The specified frequency fp is, for example, a frequency in an alpha wave band. The analysis unit 60 detects the specified frequency fp on the basis of a peak value (a local maximum value) between 8 Hz and 13 Hz in the power spectrum of the brain wave signal.

The analysis unit 60 detects, as the specified frequency fp, for example, a frequency (a peak frequency) indicating a peak value of the signal strength between 8 Hz and 13 Hz in the power spectrum. The analysis unit 60 may generate, as a result of the analysis, a signal indicating the specified frequency fp, and output the signal to the control unit 70. The specified frequency fp is a frequency in the alpha band of the individual user and may also be referred to as a peak power frequency. As described above, the analysis unit 60 identifies, as the specified frequency fp, for example, a peak frequency in the alpha wave band (from 8 Hz to 13 Hz) for each individual, and outputs the signal indicating the specified frequency fp to the control unit 70.

The control unit 70 is configured to control the content on the basis of the specified frequency of the brain wave. The control unit 70 is configured to execute processing of applying a stimulus to the user by controlling the content. In the present embodiment, the control unit 70 may control the content (such as the picture content or the audio content) to be presented to the user, on the basis of the specified frequency fp of the brain wave of the user detected by the analysis unit 60, and control the stimulus to be applied to the user.

The information processing system 1 modulates (modulates amplitude, a frequency, a phase, and the like of) the content to be provided to the user, on the basis of, for example, the specified frequency fp of the brain wave of the user. The control unit 70 may transmit, to the electronic apparatus 100, data on the content modulated on the basis of the specified frequency fp, and cause the presentation unit 20 to present the modulated content. The information processing system 1 acquires the specified frequency fp of the brain wave of the individual user to thereby set a modulation frequency of the content to match the peak frequency of the brain wave for each user, enabling application of a stimulus to the user using the content.

In the present embodiment, the control unit 70 is configured to control the content on the basis of the frequency different depending on a purpose. The control unit 70 controls the content using, as the modulation frequency, the frequency of the brain wave indicated by the specified frequency fp described above or the frequency of the brain wave estimated on the basis of the specified frequency fp.

In a case of urging the user to relax, the control unit 70 transmits, to the electronic apparatus 100, for example, data on the content modulated on the basis of a frequency of the alpha wave indicated by the specified frequency fp, and causes the presentation unit 20 to present the modulated content. In this case, the control unit 70 may transmit, to the electronic apparatus 100, image data on the picture content modulated on the basis of the frequency of the alpha wave indicated by the specified frequency fp, and cause the display 21 to display the modulated picture content. The control unit 70 may also transmit, to the electronic apparatus 100, audio data on the audio content modulated on the basis of the frequency of the alpha wave indicated by the specified frequency fp, and cause the sound output section 22 to reproduce the modulated audio content.

The information processing system 1 presents the user with the content modulated on the basis of the frequency, indicated by the specified frequency fp, of the alpha wave of the individual user, enabling application of a stimulus to the user. Controlling the content using the peak frequency, indicated by the specified frequency fp, in the alpha wave band makes it possible to effectively provide the user with a stimulus that increases the signal strength of the alpha wave. It is possible to guide the user to a relaxed state by presenting the user with content that promotes an increase in the alpha wave in the brain of the user.

In a case of urging the user to concentrate, the control unit 70 estimates a frequency of the beta wave using, for example, the frequency of the alpha wave indicated by the specified frequency fp, and transmits, to the electronic apparatus 100, data on content modulated on the basis of the estimated frequency of the beta wave. In this case, the control unit 70 may calculate, as the frequency of the beta wave, a frequency that is twice the peak frequency of the alpha wave, and output, to the electronic apparatus 100, the content modulated on the basis of the frequency of the beta wave.

The control unit 70 may transmit, to the electronic apparatus 100, image data on picture content modulated on the basis of the frequency of the beta wave, and cause the display 21 to display the modulated picture content. The control unit 70 may also transmit, to the electronic apparatus 100, audio data on audio content modulated on the basis of the frequency of the beta wave, and cause the sound output section 22 to reproduce the modulated audio content.

The information processing system 1 presents the user with the content modulated on the basis of the frequency of the beta wave, enabling application of a stimulus to the user. Controlling the content using the estimated frequency in the beta wave band makes it possible to effectively provide the user with a stimulus that increases the signal strength of the beta wave. It is possible to guide the user to a concentrated state by presenting the user with content that promotes an increase in the beta wave in the brain of the user.

Further, in a case of urging the user to sleep, the control unit 70 estimates a frequency of the theta wave using, for example, the frequency of the alpha wave indicated by the specified frequency fp, and transmits, to the electronic apparatus 100, data on content modulated on the basis of the estimated frequency of the theta wave. In this case, the control unit 70 may calculate, as the frequency of the theta wave, a frequency that is half of the peak frequency of the alpha wave, and output, to the electronic apparatus 100, the content modulated on the basis of the frequency of the theta wave.

The control unit 70 may transmit, to the electronic apparatus 100, image data on picture content modulated on the basis of the frequency of the theta wave, and cause the display 21 to display the modulated picture content. The control unit 70 may also transmit, to the electronic apparatus 100, audio data on audio content modulated on the basis of the frequency of the theta wave, and cause the sound output section 22 to reproduce the modulated audio content.

The information processing system 1 presents the user with the content modulated on the basis of the frequency of the theta wave, enabling application of a stimulus to the user. Controlling the content using the estimated peak frequency in the theta wave band makes it possible to effectively provide the user with a stimulus that increases the signal strength of the theta wave. It is possible to guide the user to a sleep state by presenting the user with content that promotes an increase in the theta wave in the brain of the user.

As described above, the information processing system 1 according to the present embodiment makes it possible to apply an appropriate stimulus to the user by detecting the specified frequency of the brain wave of the user and thereby controlling the content. The information processing system 1 makes it possible to apply a stimulus using the content in view of the peak frequency of the brain wave for each user, allowing for effectively urging the user to have a desired state.

As described above, the information processing system 1 may control the content on the basis of the frequency different depending on a purpose such as the relaxed state, the concentrated state, or the sleep state. The application of the stimulus to the user using the content makes it possible to achieve a state in which the user easily relaxes, a state in which the user easily concentrates, a state in which the user easily sleeps, or the like. It is possible to change the emotion of the user and effectively urge the user to have the relaxed state, the concentration state, or the like.

In the present embodiment, a stimulus is applied to the user using the content such as a picture or music. This configuration makes it possible to safely stimulate the brain of the user and control the state of the user to be a desired state, as compared with a case where a stimulus is applied through current application. The configuration makes it possible to measure the brain wave of the user and present the content in accordance with individual characteristics, enabling more effective application of a stimulus. The information processing system 1 makes it possible to effectively induce emotion desired by the user, through natural viewing and listening experience.

Further, in the present embodiment, the control unit 70 may estimate a frequency of a wave other than the alpha wave (such as the beta wave or the theta wave) using the peak frequency of the alpha wave indicated by the specified frequency fp, and output, to the electronic apparatus 100, data on content modulated on the basis of the estimated frequency of the wave other than the alpha wave. This configuration makes it possible to present the content modulated on the basis of the frequency of the alpha wave, the frequency of the beta wave, the frequency of the theta wave, or the like, allowing for effectively urging the user to have a desired state. The frequency of the alpha wave is accurately detectable, and the frequency of the beta wave and the frequency of the theta wave are estimated using the frequency of the alpha wave.

In the example illustrated in FIG. 2, the control unit 70 includes a content generation section 71 and a determination section 72. The content generation section 71 is configured to generate content (such as picture content or audio content) to be used to apply a stimulus such as a visual stimulus or an auditory stimulus. The content generation section 71 is configured to generate data on the content to be presented to the user, on the basis of the specified frequency fp, detected by the analysis unit 60, of the brain wave of the user.

The content generation section 71 generates, as content to be used for stimulation, the content modulated on the basis of the specified frequency fp. The content generation section 71 generates content modulated on the basis of the modulation frequency, using, as the modulation frequency, the frequency (e.g., the frequency of the alpha wave) of the brain wave indicated by the specified frequency fp or the frequency (e.g., the frequency of the beta wave or the frequency of the theta wave) of the brain wave estimated on the basis of the specified frequency fp. The content generation section 71 may generate data on content modulated on the basis of a modulation frequency different depending on a purpose such as the relaxed state or the concentrated state.

The content generation section 71 generates image data on picture content including a picture and modulated on the basis of the modulation frequency, audio data on audio content including a sound and modulated on the basis of the modulation frequency, or the like. The control unit 70 transmits, to the electronic apparatus 100, data on the content generated by the content generation section 71. It is to be noted that the content generation section 71 may newly generate content including a stimulus (such as a visual stimulus or an auditory stimulus), or may generate the content including the stimulus by processing existing content.

The content modulated by the content generation section 71 is presented to the user via the presentation unit 20, enabling application of a stimulus to the user. The application of the stimulus to the user using the content causes a change in the emotion of the user, allowing for urging the user to have the relaxed state, the concentrated state, or the like.

It is to be noted that, in a case of presenting the content including the stimulus, the content generation section 71 of the information processing system 1 may newly generate the content including the stimulus, or may generate the content including the stimulus by processing the existing content, as described above. Further, the information processing system 1 may select content suitable for the user from a library of the content including the stimulus, and present the user with the selected content.

For example, the information processing system 1 may cause the server 300 or the storage 40 of the information processing apparatus 200 to hold in advance a plurality of pieces of content different in the modulation frequency, and present the user with content that is modulated on the basis of a modulation frequency close to the peak frequency of the brain wave of the user and is selected from among the plurality of pieces of content. The control unit 70 of the information processing apparatus 200 may provide the electronic apparatus 100 with content selected from among the plurality of pieces of content held in the storage 40. The control unit 70 may also provide the electronic apparatus 100 with the content through data communication (such as downloading or streaming) with the server 300.

It is to be noted that the information processing system 1 may control the stimulus of the content by controlling hardware of the presentation unit 20, and apply the stimulus to the user. The control unit 70 may control, on the basis of the specified frequency fp, luminance of a picture outputted by the display 21 of the presentation unit 20, or may control, on the basis of the specified frequency fp, a sound in an audible band (e.g., from 4 Hz to 20000 Hz) outputted by the sound output section 22. It is to be noted that the audible band may be in a range from 20 Hz to 20000 Hz.

The control unit 70 may, for example, supply the presentation unit 20 with a signal that controls the display 21 of the presentation unit 20 on the basis of the specified frequency fp and modulate luminance of the display 21 on the basis of the specified frequency fp, thereby presenting the user with the modulated content. The control unit 70 may also supply the presentation unit 20 with the signal that controls the sound output section 22 of the presentation unit 20 on the basis of the specified frequency fp and modulate the sound in the audible band on the basis of the specified frequency fp, thereby presenting the user with the modulated content. The content of which luminance, a sound volume, or the like changes over time makes it possible to apply a stimulus to the user.

The determination section 72 of the control unit 70 is configured to determine whether or not to control the content on the basis of the specified frequency fp. The determination section 72 may determine whether or not to control the content on the basis of, for example, a brain wave signal measured after presentation of the content including the stimulus. As an example, the determination section 72 checks an effect of applying the stimulus, on the basis of an amount of a change in the signal strength (the power) in the specified frequency fp acquired by processing the brain wave signal, and determines whether or not to control the content again.

The determination section 72 grasps the amount of the change in the signal strength caused by the application of the stimulus through comparison between signal strength in the specified frequency fp before the presentation of the content and signal strength in the specified frequency fp after the presentation of the content, and determines whether or not to control the content again. After the presentation of the content including the stimulus to the user, the sensor 11 measures the brain wave and generates a brain wave signal. In a case where the display 21 displays a black image to the user after the presentation of the content including the stimulus, the sensor 11 may acquire the brain wave signal.

Further, in a case where the sound outputted by the sound output section 22 after the presentation of the content including the stimulus is silent or in a single tone, the sensor 11 may acquire the brain wave signal. Measuring the brain wave in a state in which the user is provided with a black image, a tone sound, or the like makes it possible to reduce noise mixed into the brain wave and accurately measure the brain wave (the brain wave signal).

The analysis unit 60 uses the brain wave signal acquired by the sensor 11 to calculate the signal strength in the specified frequency fp before the presentation of the content and the signal strength in the specified frequency fp after the presentation of the content. The determination section 72 makes a comparison between the signal strength in the specified frequency fp acquired by the analysis unit 60 before the presentation of the content and the signal strength in the specified frequency fp acquired by the analysis unit 60 after the presentation of the content, and checks whether or not an amount of an increase in the signal strength in the specified frequency fp is greater than or equal to a predetermined threshold.

In a case where the amount of the increase in the signal strength in the specified frequency fp is less than the predetermined threshold, the determination section 72 determines (decides) that the content including the stimulus is to be presented again. For example, in a case where the amount of the increase in the component of the alpha wave indicated by the specified frequency fp is lower than the threshold and is estimated to exhibit an insufficient effect of applying the stimulus, the determination section 72 determines that the content is to be presented again. In accordance with a result of the determination by the determination section 72, the control unit 70 causes the presentation unit 20 to present the content modulated on the basis of the frequency of the alpha wave indicated by the specified frequency fp, and applies the stimulus again to the user.

As described above, in the present embodiment, it is possible to apply the stimulus again in response to the brain wave signal measured after the presentation of the content, allowing for effectively urging the user to have a desired state. It is to be noted that the determination section 72 may check the effect of applying the stimulus, on the basis of the amount of the change in the signal strength in the frequency (e.g., the frequency of the beta wave or the frequency of the theta wave) of the brain wave estimated on the basis of the specified frequency fp, and determine whether or not to control the content again.

Further, the information processing system 1 may estimate the emotion of the user on the basis of the brain wave signal measured after the presentation of the content, and determine whether or not to control the content again, in accordance with a result of the estimation. For example, the analysis unit 60 estimates a degree of relaxation, a degree of concentration, or the like of the user using information on the component of the alpha wave, the component of the beta wave, or the like included in the brain wave signal. For example, in a case where the analysis unit 60 has estimated that the degree of relaxation is low after the presentation of the content that promotes an increase in the component of the alpha wave, the determination section 72 determines that the content that promotes the increase in the component of the alpha wave is to be presented again.

Further, for example, in a case where the analysis unit 60 has estimated that the degree of concentration is low after the presentation of the content that promotes an increase in the component of the beta wave, the determination section 72 determines that the content that promotes the increase in the component of the beta wave is to be presented again. The control unit 70 may control the content on the basis of the specified frequency fp in accordance with the result of the determination by the determination section 72, and apply the stimulus using the content.

It is to be noted that the information processing system 1 may include any other sensor than the brain wave sensor, and may estimate the emotion of the user using a signal related to the living body and acquired by the other sensor and determine whether or not to control the content again, in accordance with the result of the estimation. Examples of the other sensor include a perspiration sensor, a blood flow sensor, a myoelectric sensor, and a temperature sensor.

The analysis unit 60 estimates the emotion on the basis of a biosignal or the like indicating the brain wave, an amount of perspiration, an amount of blood, a myoelectric potential, or a skin temperature. The determination section 72 determines whether or not to present the content including the stimulus again, in accordance with the result of the estimation. The control unit 70 may apply a stimulus using the content in accordance with the result of the determination by the determination section 72.

Further, the information processing system 1 according to the present embodiment may detect a phase of the brain wave on the basis of the brain wave signal, and control the content on the basis of the specified frequency and the phase of the brain wave. For example, the analysis unit 60 is configured to detect the specified frequency and the phase of the brain wave on the basis of the brain wave signal. The control unit 70 is configured to modulate the content on the basis of the specified frequency and the phase of the brain wave of the user. The control unit 70 may present the user with the content modulated in accordance with the phase of the brain wave of the user.

For example, the control unit 70 supplies, on the basis of the detected phase of the brain wave of the user, the presentation unit 20 with a signal that controls the presentation unit 20, and controls a timing of presenting the content. The information processing system 1 presents the content including the stimulus, in synchronization with the brain wave of the user. This makes it possible to apply the stimulus using the content in view of the phase of the brain wave of the user, allowing for preventing the component of the frequency of the brain wave of the user and the component of the modulation frequency of the content from canceling each other. This makes it possible to effectively provide the user with the stimulus that increases the signal strength of the alpha wave or the like, allowing for effectively urging the user to have a desired state.

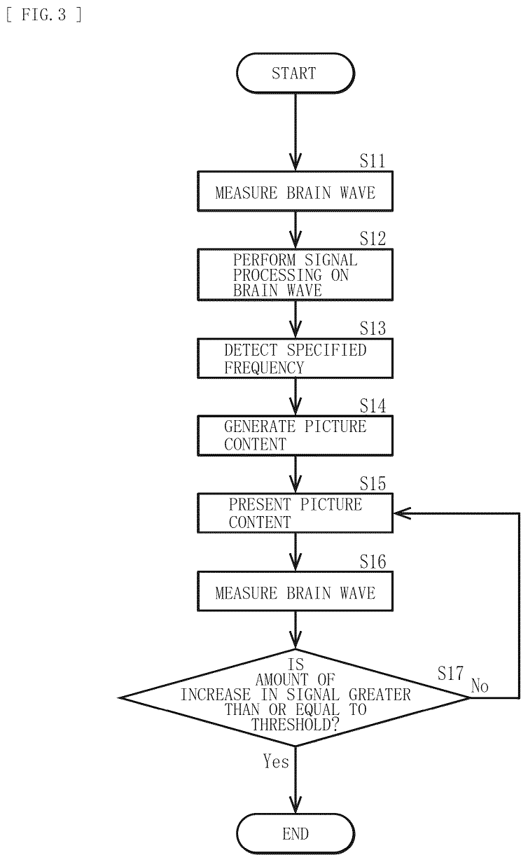

FIG. 3 is a flowchart illustrating an example of an operation of the information processing system according to the embodiment. Description is given of the example of the operation of the information processing system 1 with reference to the flowchart of FIG. 3. For example, after the application of the information processing apparatus 200 is started, the flowchart of FIG. 3 starts.

In step S11, the presentation unit 20 of the electronic apparatus 100 causes the display 21 to display a black image for a certain time period (e.g., for about 30 seconds to about 1 minute). The brain wave measurement unit 10 including the sensor 11 and incorporated into the electronic apparatus 100 (e.g., a VR device) measures the brain wave with an electrode in contact with a frontal region and an electrode in contact with an occipital region while the black image is displayed, and generates a brain wave signal. The brain wave measurement unit 10 performs AD conversion processing on the brain wave signal, and outputs, to the information processing apparatus 200, the brain wave signal converted into a digital signal.

In step S12, the analysis unit 60 of the information processing apparatus 200 performs, on the measured brain wave signal, signal processing such as band-pass filter processing of 1 Hz to 40 Hz and notch filter processing of 50 Hz/ 60 Hz commercial power supply noise. The analysis unit 60 also obtains signal strength for each frequency by performing fast Fourier transform processing on the brain wave signal, and calculates a power spectrum.

In step S13, the analysis unit 60 detects, as the specified frequency fp, a peak frequency in a range from 8 Hz to 13 Hz in the power spectrum. The analysis unit 60 outputs, to the control unit 70, a signal indicating the identified specified frequency fp.

In step S14, the content generation section 71 of the control unit 70 generates picture content (e.g., content of a CG pattern) modulated on the basis of the specified frequency fp serving as the frequency in the alpha band of the individual user. Further, the analysis unit 60 performs phase analysis processing and thereby detects a phase of the component of the modulation frequency of the brain wave.

In a case of desiring to promote the relaxation, the content generation section 71 generates the picture content modulated on the basis of the specified frequency fp serving as the frequency in the alpha band of the individual user. Further, in a case of desiring to increase the concentration, the content generation section 71 generates the picture content modulated on the basis of a frequency that is twice the specified frequency fp.

In step S15, the control unit 70 causes the presentation unit 20 to present the picture content modulated in accordance with the phase, detected by the analysis unit 60, of the brain wave. In this case, the display 21 of the presentation unit 20 presents the modulated picture content, e.g., the CG pattern, for a certain time period (e.g., for 5 minutes). It is to be noted that the presentation unit 20 may present the CG pattern with the display 21 and present modulated musical content with the sound output section 22.

In step S16, the control unit 70 causes the display 21 to present a black screen after the presentation of the content, and causes the brain wave measurement unit 10 to measure the brain wave. The control unit 70 causes the determination section 72 to make a comparison between signal strength in the specified frequency fp before the presentation of the content and signal strength in the specified frequency fp after the presentation of the content.

In step S17, the control unit 70 determines whether or not an amount of an increase in the signal strength in the specified frequency fp is greater than or equal to the predetermined threshold. In a case where the determination result in step S17 is negative (step S17: “No”), the processing returns to step S15. In step S15 to which the processing returns from step S17, the control unit 70 performs again the processing of presenting the modulated content.

In a case where the determination result in step S17 is affirmative (step S17: “Yes”), i.e., in a case where the amount of the increase in the signal strength in the specified frequency fp is greater than or equal to the predetermined threshold, the information processing system ends the processing illustrated in the flowchart of FIG. 3.

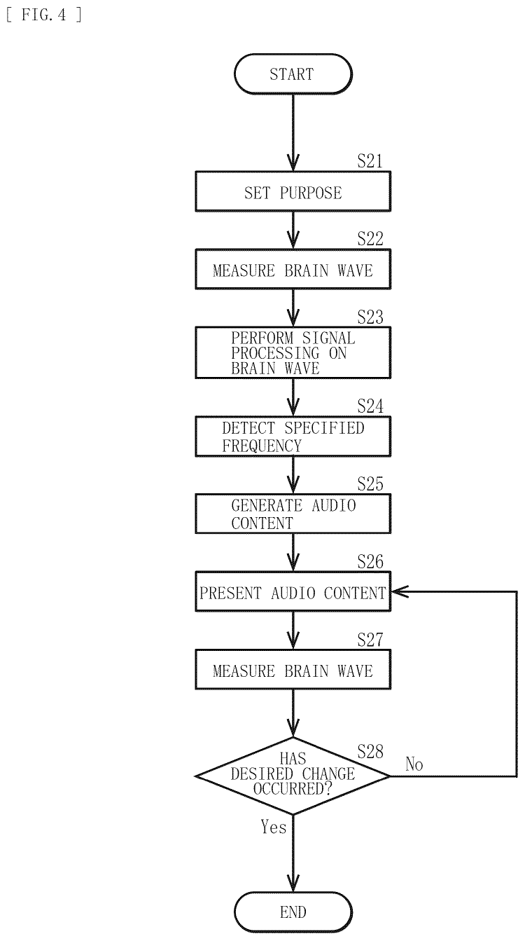

FIG. 4 is a flowchart illustrating another example of the operation of the information processing system according to the embodiment. Description is given of the example of the operation of the information processing system 1 with reference to the flowchart of FIG. 4. For example, after the application of the information processing apparatus 200 is started, the flowchart of FIG. 4 starts.

In step S21, the information processing apparatus 200 sets various control conditions by an input operation performed by the user on the operation unit 50 or through selection by the information processing apparatus 200. The purpose such as the relaxed state, the concentrated state, or the sleep state is set in the information processing apparatus 200.

In step S22, the presentation unit 20 of the electronic apparatus 100 causes the sound output section 22 to reproduce a tone of 440 Hz for a certain time period. The brain wave measurement unit 10 including the sensor 11 and incorporated into the electronic apparatus 100 (e.g., earphones) measures the brain wave using, as electrodes, electrically conductive earpieces in contact with ears while the tone is reproduced, and generates a brain wave signal. The brain wave measurement unit 10 performs AD conversion processing on the brain wave signal, and outputs, to the information processing apparatus 200, the brain wave signal converted into a digital signal.

In step S23, the analysis unit 60 of the information processing apparatus 200 performs, on the measured brain wave signal, signal processing such as band-pass filter processing of 1 Hz to 40 Hz and notch filter processing of 50 Hz/ 60 Hz commercial power supply noise. The analysis unit 60 also obtains signal strength for each frequency by performing fast Fourier transform processing on the brain wave signal, and calculates a power spectrum.

In step S24, the analysis unit 60 detects, as the specified frequency fp, a peak frequency in a range from 8 Hz to 13 Hz in the power spectrum. The analysis unit 60 outputs, to the control unit 70, a signal indicating the detected specified frequency fp.

In step S25, the content generation section 71 of the control unit 70 generates audio content (e.g., music content) modulated on the basis of the specified frequency fp serving as the frequency in the alpha band of the individual user. It is to be noted that the content generation section 71 may modulate existing music content or may automatically generate modulated audio content with AI.

The content generation section 71 generates the music content modulated in accordance with the purpose set by the operation or the like performed on the operation unit 50. For example, in a case of desiring to increase the concentration, the content generation section 71 generates the music content modulated (e.g., of which amplitude is modulated) on the basis of a frequency that is twice the specified frequency fp and corresponds to the beta wave band. Further, in a case of falling asleep, the content generation section 71 generates the music content modulated on the basis of a frequency that is half of the specified frequency fp and corresponds to the theta wave band.

In step S26, the control unit 70 causes the presentation unit 20 to present the modulated music content. For example, the presentation unit 20 presents the music content for a certain time period with the sound output section 22. It is to be noted that the presentation unit 20 may present the modulated music content with the sound output section 22 and present the modulated picture content with the display 21.

In step S27, the control unit 70 causes the sound output section 22 to reproduce a single tone of 440 Hz after the presentation of the content, and causes the brain wave measurement unit 10 to measure the brain wave. The analysis unit 60 estimates the emotion of the user on the basis of the brain wave signal and checks the state of the user.

In step S28, the control unit 70 determines whether or not to present the content again, on the basis of the result of the estimation by the analysis unit 60. In a case where the determination result in step S28 is negative (step S28: “No”), i.e., in a case where a desired change is estimated not to have occurred in the emotion of the user, the processing returns to step S26.

In step S26 to which the processing returns from step S28, the control unit 70 performs again the processing of presenting the modulated content.

In a case where the determination result in step S28 is affirmative (step S28: “Yes”), i.e., in a case where a desired change is estimated to have occurred in the emotion of the user, the information processing system ends the processing illustrated in the flowchart of FIG. 4.

### Workings and Effects

An information processing apparatus (the information processing apparatus 200) according to the present embodiment includes: an acquisition unit (the acquisition unit 30) configured to acquire a first signal (the brain wave signal) related to the brain wave of the user; an analysis unit (the analysis unit 60) configured to detect the specified frequency of the brain wave of the user on the basis of the first signal; and a control unit (the control unit 70) configured to control the content on the basis of the specified frequency.

The information processing apparatus 200 according to the present embodiment detects the specified frequency of the brain wave on the basis of the brain wave signal, and controls the content on the basis of the specified frequency. Detecting the specified frequency of the brain wave of the user and thereby controlling the content makes it possible to apply an appropriate stimulus to the user, allowing for effectively urging the user to have a desired state. This enables achievement of the information processing apparatus configured to apply a suitable stimulus.

Next, description is given of modification examples of the present disclosure. Hereinafter, the components similar to those in the foregoing embodiment are denoted by the same reference numerals, and description thereof is omitted as appropriate.

### 2. MODIFICATION EXAMPLES

### 2-1. Modification Example 1

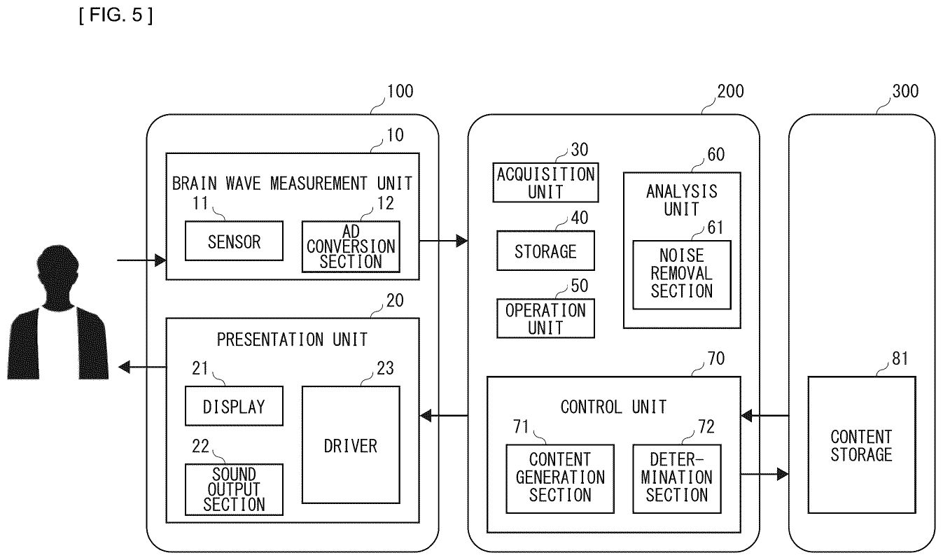

FIG. 5 is an explanatory diagram of a configuration example of an information processing system according to Modification Example 1 of the present disclosure. In the example illustrated in FIG. 5, the server 300 includes a content storage 81. The content storage 81 holds a plurality of kinds of content including a stimulus, e.g., a plurality of pieces of content different in the modulation frequency. The information processing system 1 may select content suitable for the user from a library, in the content storage 81, of the content including the stimulus, and present the user with the selected content.

The control unit 70 of the information processing apparatus 200 may receive the content from the content storage 81 via an unillustrated communication unit through data communication (such as downloading or streaming) with the server 300, and provide the electronic apparatus 100 with the received content. In this case, the control unit 70 may receive the content including the stimulus from the server 300 and transmit the received content to the electronic apparatus 100. The control unit 70 may also generate the content including the stimulus by processing the content received from the server 300, and transmit the generated content to the electronic apparatus 100.

### 2-2. Modification Example 2

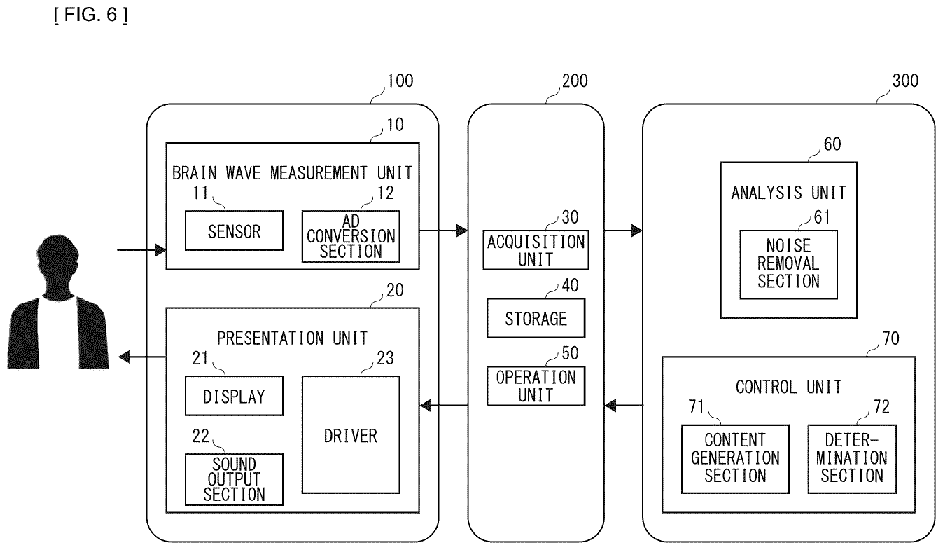

FIG. 6 is an explanatory diagram of a configuration example of an information processing system according to Modification Example 2 of the present disclosure. The foregoing embodiment describes the example in which the analysis unit 60 and the control unit 70 are provided in the information processing apparatus 200; however, some or all of the analysis unit 60 and the control unit 70 may be provided in the server 300. The information processing apparatus 200 may also serve as a relay unit, and may transmit, to the electronic apparatus 100, the content generated in an external apparatus (e.g., the server 300) on the basis of the specified frequency fp of the brain wave of the user.

### 2-3. Modification Example 3

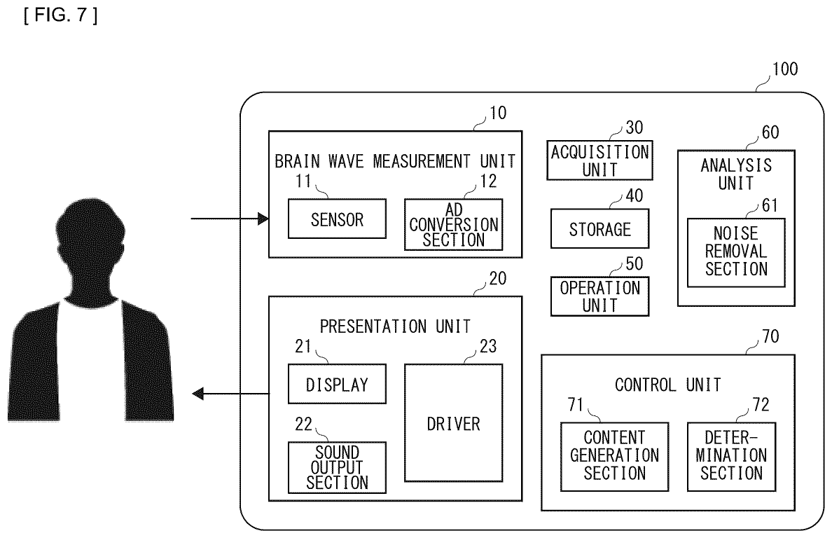

FIG. 7 is an explanatory diagram of a configuration example of an information processing system according to Modification Example 3 of the present disclosure. The analysis unit 60, the control unit 70, and the like of the information processing apparatus 200 may be provided in the electronic apparatus 100. As in the example illustrated in FIG. 7, the electronic apparatus 100 may include, for example, the acquisition unit 30, the storage 40, the operation unit 50, the analysis unit 60, and the control unit 70, in addition to the brain wave measurement unit 10 and the presentation unit 20.

### 2-4. Modification Example 4

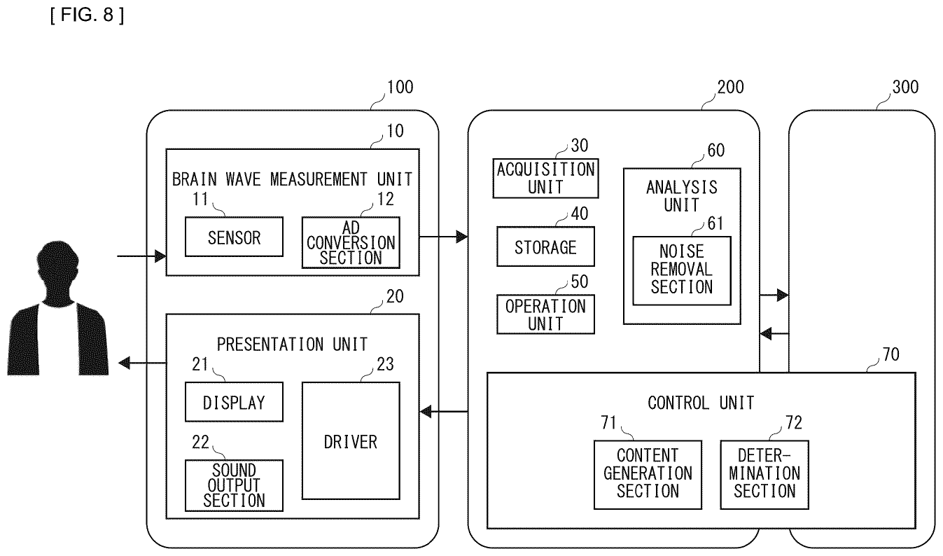

FIG. 8 is an explanatory diagram of a configuration example of an information processing system according to Modification Example 4 of the present disclosure. As in the example illustrated in FIG. 8, the control unit 70 may be provided separately in the information processing apparatus 200 and the server 300. Further, the analysis unit 60 may be provided separately in the information processing apparatus 200 and the server 300.

### 2-5. Modification Example 5

The foregoing embodiment describes the example in which the content is controlled by detecting, as the specified frequency fp, the frequency in the alpha wave band; however, the content may be controlled by detecting, as the specified frequency fp, the frequency in a band other than the alpha wave band (e.g., the frequency of the beta wave or the frequency of the theta wave).

For example, the analysis unit 60 may detect, as the specified frequency fp, a frequency (a peak frequency) indicating a peak value of the signal strength between 13 Hz and 30 Hz in the power spectrum. The analysis unit 60 may also detect, as the specified frequency fp, a peak frequency indicating a peak value of the signal strength between 4 Hz and 8 Hz in the power spectrum. The control unit 70 may control the content by using, as the modulating frequency, the frequency (e.g., the peak frequency of the beta wave or the peak frequency of the theta wave) of the brain wave indicated by the specified frequency fp or the frequency of the brain wave estimated on the basis of the specified frequency fp.

### 2-6. Modification Example 6

The series of pieces of the processing described in the foregoing embodiment and modification examples is executable by hardware, software, or a combination of both. In a case where the processing is executed by software, a program having recorded a processing sequence is executable after being installed in a memory that is provided in a computer and incorporated into dedicated hardware.

The program is also executable after being installed in a general-purpose computer configured to execute various kinds of processing. For example, it is possible to record in advance the program on a recording medium. In addition to being installed in a computer from a recording medium, the program is also installable in a recording medium such as a built-in hard disk after being received via a network such as a LAN (Local Area Network) or the Internet.

As described above, it is possible to cause the computer (the processor) to execute the program that performs the processing according to the present disclosure. A technique according to the present disclosure is also achievable as a computer program product.

Although the present disclosure has been described above with reference to the embodiment and the modification examples, the present technology is not limited to the foregoing embodiment and the like, and may be modified in a variety of ways. For example, the foregoing modification examples have been described as modification examples of the foregoing embodiment, but are achievable by any combination of configurations of the respective modification examples as appropriate. Further, although a human body has been described as an example of the living body, the present disclosure is also applicable to any other living body than the human body, e.g., an animal such as a pet or a domestic animal.

An information processing apparatus according to an embodiment of the present disclosure includes: an acquisition unit configured to acquire a first signal related to a brain wave of a user; an analysis unit configured to detect a specified frequency of the brain wave of the user on the basis of the first signal; and a control unit configured to control content on the basis of the specified frequency. Such a configuration makes it possible to apply an appropriate stimulus to the user. This enables achievement of the information processing apparatus configured to apply a suitable stimulus.

It is to be noted that the effects described herein are merely exemplary and are not limited to the description, and any other effects may be obtained. Further, the present disclosure may have the following configurations.
(1)

An information processing apparatus including:
an acquisition unit configured to acquire a first signal related to a brain wave of a user;
an analysis unit configured to detect a specified frequency of the brain wave of the user on a basis of the first signal; and
a control unit configured to control content on a basis of the specified frequency.
(2)

The information processing apparatus according to (1), in which the control unit is configured to execute processing of controlling the content and applying a stimulus to the user.
(3)

The information processing apparatus according to (1) or (2), in which the control unit is configured to control the content on a basis of a frequency that is different depending on a purpose.
(4)

The information processing apparatus according to any one of (1) to (3), in which
the content includes picture content, and
the control unit is configured to control luminance of a picture on a basis of the specified frequency.
(5)

The information processing apparatus according to any one of (1) to (3), in which
the content includes audio content, and
the control unit is configured to control amplitude modulation in an audible band on a basis of the specified frequency.
(6)

The information processing apparatus according to any one of (1) to (5), in which the specified frequency includes a frequency of an alpha wave.
(7)

The information processing apparatus according to any one of (1) to (6), in which the analysis unit is configured to detect the specified frequency on a basis of a peak value that is between 8 Hz and 13 Hz in a power spectrum of the first signal.
(8)

The information processing apparatus according to any one of (1) to (7), in which the analysis unit is configured to detect a phase of the brain wave of the user on a basis of the first signal, and
the control unit is configured to modulate the content on a basis of the specified frequency of the brain wave of the user and the phase of the brain wave of the user.
(9)

The information processing apparatus according to any one of (1) to (8), in which
the analysis unit is configured to perform analysis of the first signal that is acquired by the acquisition unit in a case where the content has been presented, and
the control unit is configured to control the content on the basis of the specified frequency in accordance with a result of the analysis performed by the analysis unit.
(10)

The information processing apparatus according to any one of (1) to (9), in which the analysis unit is configured to perform estimation of emotion of the user on a basis of the first signal.
(11)

The information processing apparatus according to (10), in which the control unit is configured to control the content on the basis of the specified frequency in accordance with a result of the estimation performed by the analysis unit.
(12)

The information processing apparatus according to any one of (1) to (11), including a sensor configured to generate the first signal, in which
the acquisition unit is configured to acquire the first signal from the sensor.
(13)

An information processing system including:
an electronic apparatus including a sensor, the sensor being configured to generate a first signal related to a brain wave of a user; and
an information processing apparatus including
   an analysis unit configured to detect a specified frequency of the brain wave of the user on a basis of the first signal, and
   a control unit configured to control content on a basis of the specified frequency.
(14)

The information processing system according to (13), in which the electronic apparatus includes a wearable device.
(15)

The information processing system according to (13) or (14), including a display configured to display an image, in which
the sensor is configured to generate the first signal in a case where the display displays a black image.
(16)

The information processing system according to any one of (13) to (15), including a sound output section configured to output a sound, in which
the sensor is configured to generate the first signal in a case where the sound outputted by the sound output section is silent or in a single tone.
(17)

An information processing program causing a computer to:
acquire a first signal related to a brain wave of a user;
detect a specified frequency of the brain wave of the user on a basis of the first signal; and
control content on a basis of the specified frequency.

The present application claims the benefit of Japanese Priority Patent Application JP2022-130794 filed with the Japan Patent Office on Aug. 18, 2022, the entire contents of which are incorporated herein by reference.

It should be understood by those skilled in the art that various modifications, combinations, sub-combinations and alterations may occur depending on design requirements and other factors insofar as they are within the scope of the appended claims or the equivalents thereof.

1. An information processing apparatus comprising:
an acquisition unit configured to acquire a first signal related to a brain wave of a user;
an analysis unit configured to detect a specified frequency of the brain wave of the user on a basis of the first signal; and
a control unit configured to control content on a basis of the specified frequency.

2. The information processing apparatus according to claim 1, wherein the control unit is configured to execute processing of controlling the content and applying a stimulus to the user.

3. The information processing apparatus according to claim 1, wherein the control unit is configured to control the content on a basis of a frequency that is different depending on a purpose.

4. The information processing apparatus according to claim 1, wherein
the content comprises picture content, and
the control unit is configured to control luminance of a picture on a basis of the specified frequency.

5. The information processing apparatus according to claim 1, wherein
the content comprises audio content, and
the control unit is configured to control amplitude modulation in an audible band on a basis of the specified frequency.

6. The information processing apparatus according to claim 1, wherein the specified frequency comprises a frequency of an alpha wave.

7. The information processing apparatus according to claim 1, wherein the analysis unit is configured to detect the specified frequency on a basis of a peak value that is between 8 Hz and 13 Hz in a power spectrum of the first signal.

8. The information processing apparatus according to claim 1, wherein
the analysis unit is configured to detect a phase of the brain wave of the user on a basis of the first signal, and
the control unit is configured to modulate the content on a basis of the specified frequency of the brain wave of the user and the phase of the brain wave of the user.

9. The information processing apparatus according to claim 1, wherein
the analysis unit is configured to perform analysis of the first signal that is acquired by the acquisition unit in a case where the content has been presented, and
the control unit is configured to control the content on the basis of the specified frequency in accordance with a result of the analysis performed by the analysis unit.

10. The information processing apparatus according to claim 1, wherein the analysis unit is configured to perform estimation of emotion of the user on a basis of the first signal.

11. The information processing apparatus according to claim 10, wherein the control unit is configured to control the content on the basis of the specified frequency in accordance with a result of the estimation performed by the analysis unit.

12. The information processing apparatus according to claim 1, comprising a sensor configured to generate the first signal, wherein
the acquisition unit is configured to acquire the first signal from the sensor.

13. An information processing system comprising:
an electronic apparatus including a sensor, the sensor being configured to generate a first signal related to a brain wave of a user; and
an information processing apparatus including
an analysis unit configured to detect a specified frequency of the brain wave of the user on a basis of the first signal, and
a control unit configured to control content on a basis of the specified frequency.

14. The information processing system according to claim 13, wherein the electronic apparatus comprises a wearable device.

15. The information processing system according to claim 13, comprising a display configured to display an image, wherein
the sensor is configured to generate the first signal in a case where the display displays a black image.

16. The information processing system according to claim 13, comprising a sound output section configured to output a sound, wherein
the sensor is configured to generate the first signal in a case where the sound outputted by the sound output section is silent or in a single tone.

17. An information processing program causing a computer to:
acquire a first signal related to a brain wave of a user;
detect a specified frequency of the brain wave of the user on a basis of the first signal; and
control content on a basis of the specified frequency.
